(19) United States
(12) Patent Application Publication
(10) Pub. No.: US 2011/0046549 A1
(43) Pub. Date: Feb. 24, 2011

(54) ENDOLUMINAL SURGERY CATHETER

(75) Inventors: Igor Waysbeyn, Haifa (IL); Irina Kavounovski, Haifa (IL); Itzhak Koifman, Haifa (IL)

Correspondence Address: William H. Dippert;Eckert Seamans Cherin & Mellott, LLC, U.S. Steel Tower, 600 Grant Street, 44th Floor, Pittsburgh, PA, 15219, US

(73) Assignee: MEDIVUR LTD., Haifa (IL)

(21) Appl. No.: 12/742,882

(22) PCT Filed: Nov. 16, 2008
(86) PCT No.: PCT/IL2008/001503

§ 371 Date: Nov. 2, 2010

Related U.S. Application Data
(60) Provisional application No. 60/996,368, filed on Nov. 14, 2007.

(51) Int. Cl. A61M 29/00; A61M 1/00

(52) U.S. Cl. 604/101.01; 604/129

(57) ABSTRACT

The disclosed invention provides a system and method for accurately and in a controllable manner removing layers of tissue, deposits and/or plaque off surfaces of body is cavity for example a blood vessel. According to an aspect of the current invention, a catheter (10) endarterectomy having a proximal end (12) and a distal end (14) is provided, the catheter comprising: a pair of inflatable balloons (16) disposed at said distal end, wherein a predefined distance separate between the centers of said balloons; said scraper (18) having at least one blade (40) disposed between said balloons, wherein said scraper is reciprocally movable between two positions one of which, which is the first position, is distally disposed to the other position, which is the second position, and at least one vacuum aperture (39) disposed adjacent to said at least one blade.

This present application claims the benefit of earlier U.S. provisional patent application Ser. No. 60/996,368 filed on Nov. 14, 2007 by Igor Waysbeyn et al. and entitled “Catheter for Endarterectomy”.

### FIELD OF THE INVENTION

The present invention relates in general to a method and apparatus for endoluminal surgery. More particularly, the invention relates to a method and apparatus for removing layers of tissue, plaque, including fatty deposits and other occlusions from surfaces of vessels and/or cavities within mammalian bodies.

### BACKGROUND OF THE INVENTION

Endarterectomy procedures are known. US patent application 20020029052A1 discloses systems, methods and kits for endarterectomy procedures. A set of catheters and tools, such as catheter having one or two inflatable balloons, guiding wires, blades and extendable blades are provided. The tools provide for dissecting through the innermost tissue layer of the lumen of a body cavity or an endoluminal tract within the human body to an underlying tissue layer. In accordance with the disclosed method the innermost tissue layer is then stripped away from the underlying layer including the occlusive material. In accordance with this disclosure the detached tissue layer and occlusive material is then removed from the lumen optionally with additional cutting, maceration and suctioning.

In U.S. Pat. No. 5,188,595 a catheter retainable in place by means of a pair of inflatable balloons is disclosed. The balloons are circumferentially disposed at the distal end of the catheter. One or two fluid passageways within the catheter provide for inflating or deflating the balloons. Additional fluid passageway having an aperture disposed between the balloons provides for applying vacuum in the volume enclosed between the balloons and the walls of the body cavity thereby securing the catheter in place within its lumen.

Thus, there is a need for a system and a method providing for retaining an endarterectomy catheter in place providing for an accurate and controllable operation for the removing of layers of tissue, deposits and plaque off the inner surfaces of body cavities.

### SUMMARY OF THE INVENTION

The present invention successfully addresses the shortcomings of the presently known configurations by providing devices and methods for retaining an endarterectomy catheter in place providing for an accurate and controllable operation for the removing of layers of tissue, deposits and plaque off the inner surfaces of body cavities.

According to an aspect of the current invention, a catheter for endarterectomy (CE) having a proximal end and a distal end is provided, said CE comprising: a pair of inflatable balloons disposed at said distal end, wherein a predefined distance separate between the centers of said balloons; a scraper having at least one blade disposed between said balloons, wherein said scraper is reciprocally movable between two positions one of which, which is the first position, is distally disposed to the other position, which is the second position, and at least one vacuum aperture disposed adjacent to said at least one blade.

In some embodiments the CE comprises a fluid passageway connecting between the lumen of at least one of said balloons and an inflating/deflating port disposed at said proximal end.

In some embodiments the vacuum passageway connects between said at least one vacuum aperture and a vacuum port disposed at said proximal end.

In some embodiments the CE further comprising a distal aperture disposed at said distal end, and wherein a by-pass fluid passageway connects between said distal aperture and a by-pass port proximally disposed to said pair of balloons.

In some embodiments the segment of the surface of said fluid passageway is coated with an anticoagulant.

In some embodiments the blade comprises a beveled tip.

In some embodiments the blade is proximally skewed.

In some embodiments the AC is further provided with a vacuum source connected to said vacuum port such that the pressure within a space confined with segments of the external surface of said balloons is controllable.

According to another aspect of the current invention, a method for removing a layer from a targeted segment of a wall of a cavity disposed within mammalian body by means of a catheter for endarterectomy (CE) having a distal end and a proximal end, wherein said CE has a pair of circumferential inflatable balloons disposed at its distal end is provided, said method comprising the steps of: introducing said CE into said cavity and placing it such that said segment of wall is positioned between said balloons; inflating said balloons, such that both balloons press the wall of said body cavity at a pressure level exceeding a predefined threshold; inwardly bending said segment of wall by lowering the pressure within the volume enclosed by segments of the surfaces of said balloons and said segment of wall to a predefined pressure level; and proximally pulling a scraper from a first position towards a second position, and wherein said CE comprises said scraper, and wherein said scraper comprises at least one blade against which said inwardly bended segment of wall presses, and wherein said scraper is reciprocally movable between said first position and said second position, and wherein said first position is distally disposed along said CE relative to said second position.

In some embodiments the method further comprising moving said scraper to the first position prior to said lowering.

In some embodiments the method further comprising the steps of: distally moving said scraper to said first position; lowering the pressure within said volume by a predefined level; and pulling said scraper towards said second position, and wherein said above mentioned steps follow said step of proximally pulling a scraper from a first position towards a second position.

In some embodiments the method further comprises suctioning debris off said volume.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 schematically depicts a sectional view of a catheter for endarterectomy according to a preferred embodiment of the present invention;

FIG. 2 schematically depicts a detailed sectional view of a segment of the catheter shown in FIG. 1 placed within a body cavity;

### DETAILED DESCRIPTION OF THE PRESENT INVENTION

In accordance with the present invention a system and method for accurately and in a controllable manner removing layers of tissue, deposits and/or plaque off surfaces of natural and/or artificial cavity within mammalian body is provided. The catheter of the invention provides for securing its placement at a targeted location within either natural or artificially structured body cavity; accurately controlling the widths of layers to be dissected and removed from the wall of the cavity, and suctioning off debris of tissue, plaque and/or deposits from the lumen of the cavity. The catheter of the invention provides for a minimal invasive surgery, such as dissecting and removing off a segment of tissue disposed within an organ as well as for endarterectomy. The catheter of the invention is referred hereinafter as catheter for endarterectomy.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

In discussion of the various figures described herein below, like numbers refer to like parts. The drawings are generally not to scale.

For clarity, non-essential elements were omitted from some of the drawings.

As used herein, an element or step recited in the singular and proceeded with the word “a” or “an” should be understood as not excluding plural elements or steps, unless such exclusion is explicitly recited.

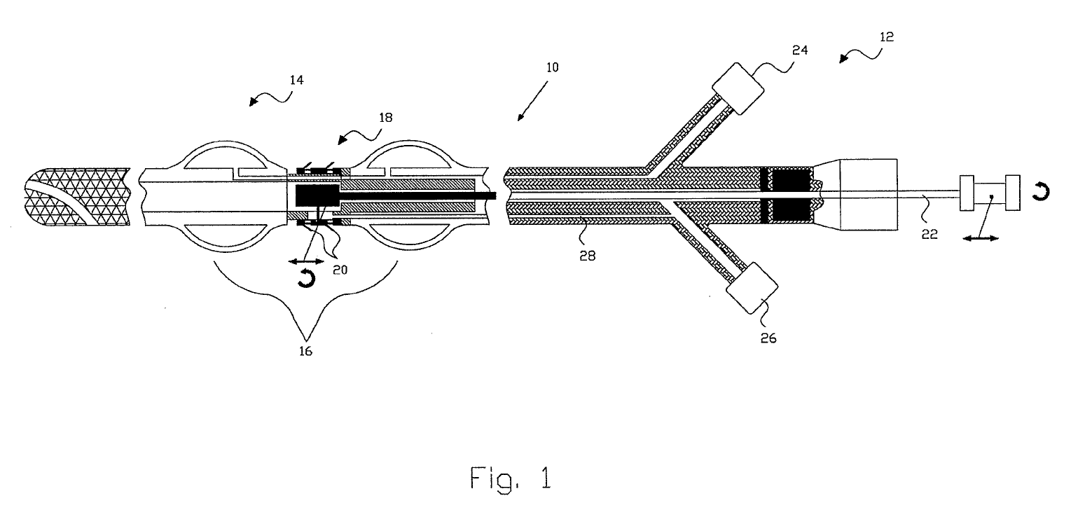

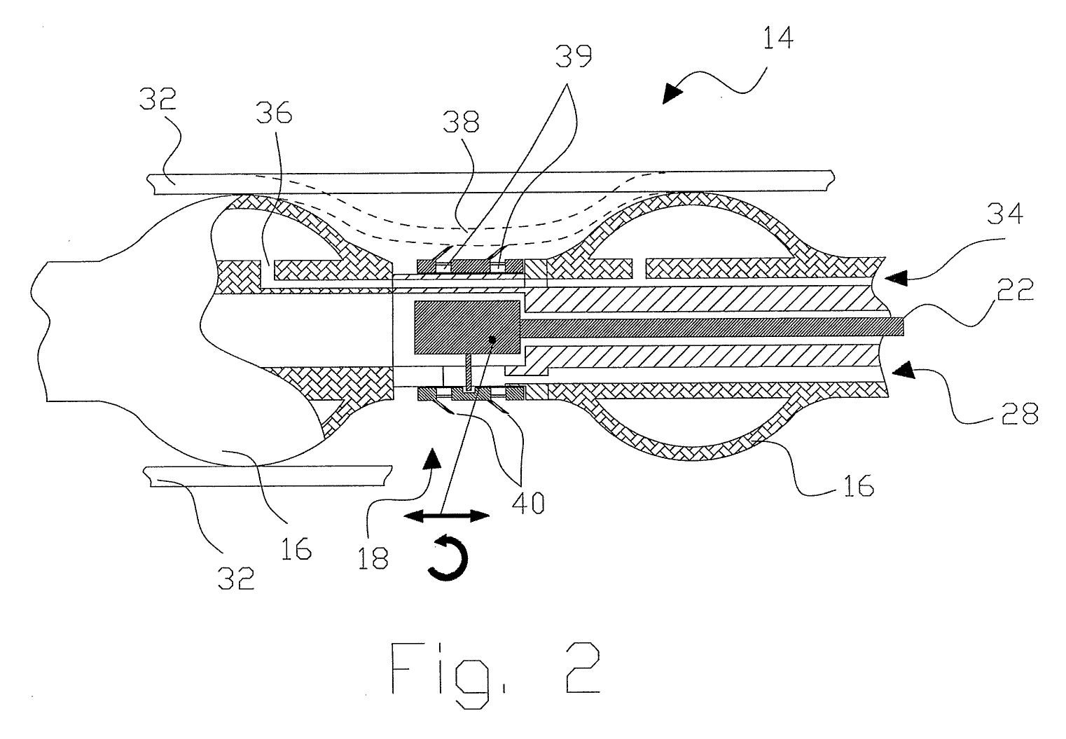

Reference is made to FIGS. 1-2. In FIG. 1 a sectional view of a Catheter for Enarterectomy (CE) in accordance with a preferred embodiment of the present invention is shown. CE 10 has proximal end 12 and distal end 14. A pair of circumferential inflatable balloons 16 is disposed at distal end 14. The distance separating between the balloons is predefined. Scraper 18 having at least one blade, such as blades 20 is disposed between balloons 16. Scraper 18 is reciprocally distally and proximally movable by means of activating wire 22, which is engaged with the scraper. Inflating/deflating port 24 provides for flowing fluids through for respectively inflating and/or deflating balloons 16. Optionally, each balloon of the pair is respectively connected to a separate inflating/deflating port 24 disposed at proximal end 12, thereby inflating or deflating of the balloons is respectively accomplished independently. In FIG. 2 a vacuum port 26 is connected by fluid passageway 28 to one or more vacuum apertures 39 disposed adjacent to blades 20. The vacuum apertures provide for inducing vacuum within a volume confined by both balloons 16 and an enclosing surface 32 of the body cavity into which CE 10 is introduced.

In FIG. 2 a sectional view of distal end 14 of the CE shown in FIG. 1 is schematically shown being introduced into a tubular body cavity enclosed by wall 32. Balloons 16 are inflated by pressurizing fluid through fluid passageway 34 and apertures 36 into their respective lumens. By connecting the respective inflating/deflating port 26 disposed at the proximal end 12 of the catheter to a source of vacuum, vacuum is induced within the space enclosed between the balloons and wall 32. The reduced pressure inwardly bends a segment of the wall, such as segment 38. Such bended segment of the wall provides for securing distal end 14 in place within the body cavity. Such securing provide for inhibiting translation or axial rotation of the distal end of the CE relative to wall 32. Furthermore segment 38 presses against the tips of blades 40 of scraper 18. The level of the vacuum applied, the distance between the centers of balloons 16 and their radii as well as the level of the blades' tips relative to the lateral width of the cavity determine the level of force in which segment 38 presses against the tips of blades 40. Blades 40 are optionally proximally skewed and each of them optionally has a beveled tip. Therefore by proximally puling and pushing activating wire 22 in linear movements as well as rotational movements that are shown by the arrows in FIG. 1, a respective layer is dissected and peeled off segment 38. The movements of the blades are also shown by arrows in FIG. 2. In accordance with the method of the present invention the depth in which segment 38 is dissected by such proximally pulling and rotating of the scraper is controlled by fine tuning of the pressure within the volume enclosing the targeted segment to be peeled.

For operating a CE of the invention first a catheter having a suitable distance separating between the centers of its balloons and a predefined length of blades, such that the level of the blades' tips is inwardly disposed by a predefined distance relative to the topmost level of the inflated balloons, is selected. Then the catheter is introduced into the body cavity and placed such that the distal balloon is distally disposed and the proximal balloon is proximally disposed to the targeted location. The activating wire 22 is distally pushed to its distal position. Then the balloons are inflated by pressurizing fluid into their lumens up to a pressure which is not lower than a threshold. This threshold is such selected that the balloons press the cavity's wall at a predefined pressure. Such pressure provides, for example, the sealing off the fluid passageways between the volume confined between the balloons and the rest of the cavity in both proximal and distal directions. Namely, any bleeding, due to perforation of the wall, through a defected wall of an exemplary operated blood vessel is practically prevented. Vacuum is induced at a first predefined level within the volume enclosed by the balloons and the segment of the cavity's wall in the following step. Then the activating wire 22 is proximally pulled along either a predefined distance or up to its maximal extent, thereby a layers of a first width are dissected and peeled off segment 38. Optionally the activating wire is distally pushed to its maximal distal position, the level of the vacuum is increased to reach a second predefined pressure and the dissecting of another layer is repeated again. The entire process of lowering the pressure and pulling the activating wire is repeated until the obstruction or the full layer of tissue to be peeled off is removed. Debris are suctioned by the vacuum applied through lumen 28. Optionally the volume of the cavity enclosed within the pair of balloons is rinsed for an improved removal of the debris. Furthermore, rinsing is optionally accomplished by utilizing a solution having a predefined composition, such as for decaying a future stenosis.

Embodiments variants in which an additional fluid passageway connects between an aperture disposed at the distal end of the catheter and a port disposed proximally to the proximal balloon, providing for by-passing the segment of the body cavity enclosed between the balloons are in accordance with the present invention.

It should be mentioned that optionally, a guide wire as known in the art can be inserted through the catheter or the catheter tip (as shown in FIG. 1) can be used in order to direct the catheter to the proper place.

Typically catheters of the invention are made of biocompatible polymer materials. The blades are typically made of stainless steel. However other metals or rigid plastic resins may serve as well for manufacturing the blades, such as by molding. The segment of the catheter confined between both balloons is optionally made of relatively thick polymer material and is further preferably reinforced by embedded rigid netting or other means providing for an enhanced axial stiffness. Optionally surfaces exposed to inner liquids such as blood are further coated with an anticoagulant for decaying blood clotting as known.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A catheter for endarterectomy (CE) having a proximal end and a distal end, said CE comprising:
a pair of inflatable balloons disposed at said distal end, wherein a predefined distance separate between the centers of said balloons;
a scraper having at least one blade disposed between said balloons, wherein said scraper is reciprocally movable between two positions one of which, which is the first position, is distally disposed to the other position, which is the second position,
at least one vacuum aperture disposed adjacent to said at least one blade.

2. A CE as in claim 1, wherein a fluid passageway connects between the lumen of at least one of said balloons and an inflating/deflating port disposed at said proximal end.

3. A CE as in claim 1, wherein a vacuum passageway connects between said at least one vacuum aperture and a vacuum port disposed at said proximal end.

4. A CE as in claim 1 further comprising a distal aperture disposed at said distal end, and wherein a by-pass fluid passageway connects between said distal aperture and a by-pass port proximally disposed to said pair of balloons.

5. A CE as in claim 4, wherein a segment of the surface of said fluid passageway is coated with an anticoagulant.

6. A CE as in claim 1, wherein said at least one blade comprises a bevelled tip.

7. A CE as in claim 1, wherein said at least one blade is proximally skewed.

8. A CE as in claim 3, in combination with a vacuum source connected to said vacuum port such that the pressure within a space confined with segments of the external surface of said balloons is controllable.

9. A CE as in claim 1, wherein said scrapper is capable of rotational movement, to provide pulverization of the dissected wall layer.

10. A method for removing a layer from a targeted segment of a wall of a cavity disposed within human body by means of a catheter for endarterectomy (CE) having a distal end and a proximal end, wherein said CE has a pair of circumferential inflatable balloons disposed at its distal end, said method comprising the steps of:
introducing said CE into said cavity and placing it such that said segment of wall is positioned between said balloons;
inflating said balloons, such that both balloons press the wall of said body cavity at a pressure level exceeding a predefined threshold;
inwardly bending said segment of wall by lowering the pressure within the volume enclosed by segments of the surfaces of said balloons and said segment of wall to a predefined pressure level; and
proximally pulling a scraper from a first position towards a second position, and
wherein said CE comprises said scraper, and wherein said scraper comprises at least one blade against which said inwardly bended segment of wall presses, and wherein said scraper is reciprocally movable between said first position and said second position, and wherein said first position is distally disposed along said CE relative to said second position.

11. A method as in claim 9 further comprising moving said scraper to the first position prior to said lowering.

12. A method as in claim 10, further comprising the steps of:
distally moving said scraper to said first position;
lowering the pressure within said volume by a predefined level; and
pulling said scraper towards said second position.

13. The method as claimed in claim 10, further comprising rotating said scrapper.

14. A method as in any of claim 10 or 11, further comprising suctioning debris of said volume.

15. A method as claimed in claim 10, further comprising the step of removing the CE from the body cavity with slightly inflated balloons and the remains of said debris.
